(19) United States
(12) Patent Application Publication
(10) Pub. No.: US 2025/0146142 A1
(43) Pub. Date: May 8, 2025

(54) Method for generating synthesis gas for use in hydroformylation reactions

(71) Applicant: HALDOR TOPSØE A/S

(72) Inventors: Niels Christian SCHJØDT, Hvalsø (DK); Rainer KÜNGAS, Copenhagen (DK); Berit HINNEMANN, Stenløse (DK); Bengt Peter Gustav BLENNOW, Humlebæk (DK)

(73) Assignee: HALDOR TOPSØE A/S, Kgs. Lyngby (DK)

(21) Appl. No.: 18/987,417

(22) Filed: Dec. 19, 2024

Related U.S. Application Data
(63) Continuation of application No. 17/046,049, filed on Oct. 8, 2020, now Pat. No. 12,215,432.

(30) Foreign Application Priority Data
Apr. 13, 2018 (DK) PA 2018 00156

(51) Int. Cl. C25B 1/23; C01B 3/06; C01B 32/40; C10K 1/04; C10K 3/02; C25B 1/042; C25B 9/19; C25B 9/70; C25B 13/05; C25B 13/07; C25B 15/021; C25B 15/08

(52) U.S. Cl. CPC C25B 1/23; C01B 3/06; C01B 32/40; C10K 1/04; C10K 3/026; C25B 1/042; C25B 9/19; C25B 9/70; C25B 13/05; C25B 13/07; C25B 15/021; C25B 15/08; C25B 15/081; C25B 15/083; C01B 2203/02; C01B 2203/0495; C01B 2203/06; C01B 2203/12

(57) ABSTRACT

A method for the generation of a gas mixture including carbon monoxide, carbon dioxide, and hydrogen for use in hydroformylation plants, including: evaporating water to steam; feeding the steam to a solid oxide electrolysis cell (SOEC) while supplying an electrical current to the SOEC to effect a partial conversion of steam to hydrogen; utilizing the effluent SOEC gas including H₂ together with CO₂ from an external source as feed for a RWGS reactor in which the RWGS reaction takes place, converting some of the CO₂ and H₂ to CO and H₂O; removing some of or all the remaining steam from the raw product gas stream by cooling the raw product gas stream allowing for condensation of at least part of the steam as liquid water and separating the remaining product gas from the liquid; using the gas mixture for liquid phase hydroformylation, while recycling CO₂ to the RWGS reactor.

### CROSS REFERENCE TO RELATED APPLICATIONS

The present application is a continuation of U.S. patent application Ser. No. 17/046,049, filed on Oct. 8, 2020, which is a U.S. National Stage of International Application No. PCT/EP2019/059201, filed on Apr. 11, 2019, which claims the benefit of Danish Application No. PA 2018 00156, filed on Apr. 13, 2018. The entire contents of U.S. patent application Ser. No. 17/046,049, International Application No. PCT/EP2019/059201, and Danish Application No. PA 2018 00156 are hereby incorporated herein by reference in their entirety.

### TECHNICAL FIELD

The present invention relates to a method for generating a gas comprising carbon monoxide and hydrogen and its use in hydroformylation reactions.

### BACKGROUND

Hydroformylation, also known as “oxo synthesis” or “oxo process”, is an industrial process for the production of aldehydes from alkenes. More specifically, the hydroformylation reaction is the addition of carbon monoxide (CO) and hydrogen (H₂) to an alkene. This chemical reaction entails the net addition of a formyl group (CHO) and a hydrogen atom to a carbon-carbon double bond. The reaction yields an aldehyde with a carbon chain one unit longer than that of the parent alkene. If the aldehyde is the desired product, then the syngas should have a composition close to CO:H₂=1:1.

In some cases, the alcohol corresponding to the aldehyde is the desired product. When this is the case, more hydrogen is consumed to reduce the intermediate aldehyde to an alcohol, and therefore the syngas should have a composition of approximately CO:H₂=1:2.

Sometimes it is desired to purify the intermediate aldehyde before converting it into an alcohol. Accordingly, in such case, a syngas with the composition CO:H₂=1:1 must first be used, followed by pure H₂.

Thus, the need for low-module syngas (i.e. syngas with a low hydrogen-to-carbon monoxide ratio) is characteristic for the hydroformylation reaction. Such syngas compositions are rather costly to provide since they cannot be obtained directly from steam reforming of natural gas or naphtha. At least, a cold box for condensing CO has to be installed to separate the CO. This is a costly solution, and there will be an excess of hydrogen for which a use purpose has to be found.

Alternatively, gasification plants may provide low-module syngas, but gasification plants need to be very large to be efficient, and they are expensive, both with respect to CAPEX and to OPEX. Furthermore, coal-based gasification plants are increasingly undesired due to the substantial environmental implications and a large CO₂ footprint.

Low-module (i.e. CO-rich) syngas for hydroformylation is therefore generally costly. Large hydroformylation plants are often placed in industrial areas and may thus obtain the necessary syngas “over the fence” from a nearby syngas producer. In many cases, however, this is not possible for medium or small size hydroformylation plants. Instead, such smaller plants will need to import the syngas, e.g. in gas cylinders, and this approach is very expensive. Furthermore, transportation and handling of such gas containers is connected with certain elements of risk since syngas (not least low-module syngas) is highly toxic and extremely flammable, and syngas may form explosive mixtures with air. Import of CO or syngas by tube trailers will face similar challenges, both in terms of costs and in terms of safety.

A sustainable source of CO is CO₂. By means of the reverse water gas shift (RWGS) reaction, i.e. the reaction

CO₂+H₂<->CO+H₂O

carbon monoxide can be generated from carbon dioxide. The hydrogen used for the reaction can be generated from steam using a solid oxide electrolysis cell (SOEC) or an SOEC stack. According to the present invention, H₂ is generated from steam in an SOEC or an SOEC stack at an elevated temperature, typically close to 700° C. The effluent gas from the SOEC or SOEC stack will contain H₂ and H₂O at a temperature close to the operating temperature of the SOEC. This effluent gas may be led directly to a RWGS reactor together with CO₂. The RWGS reactor is typically a heated reactor but may also be an adiabatic reactor. In the RWGS reactor, the above RWGS reaction proceeds. Since the RWGS reaction is equilibrium limited, the effluent gas from the RWGS reactor will contain H₂, CO, H₂O and CO₂. Water is removed by cooling the effluent gas until the majority of the steam condenses as liquid water. Further drying of the gas may be achieved by using e.g. a drying column. The CO₂ can be removed by using e.g. a pressure swing adsorption (PSA) apparatus, but such apparatus is very costly. According to the present invention, CO₂ is not removed until after the hydroformylation reaction. This is cost saving and presents an additional advantage.

It has been shown for the hydroformylation reaction that the rate may be increased up to four-fold by conducting the reaction in CO₂-expanded liquid (CXL) media (see e.g. H. Jin and B. Subramaniam, Chemical Engineering Science 59 (2004) 4887-4893 and H. Jin et al., AlChE Journal 52 (2006) 2575-2581). Pressurizing an organic solvent with CO₂ makes it expand and increases the diffusivity and solubility of other (reactant) gases compared to the neat solvent. The use of CXL media is a general way of intensifying liquid phase catalytic reactions, such as carbonylation reactions. However, a source of CO₂ as well as a source of CO and a source of H₂ is needed, which is not always feasible. The present invention is aimed at solving these problems.

### SUMMARY

So the present invention relates to a novel method for the generation of a gas mixture comprising carbon monoxide, carbon dioxide and hydrogen for use in hydroformylation plants. Through the method of the invention, the above problems combined are turned into an advantage. By combining high-temperature electrolysis of steam (forming hydrogen) with the RWGS reaction (forming carbon monoxide), a low-module syngas may be obtained. Thus, carbon dioxide will serve as the sole source of carbon monoxide, and therefore, any storage, transportation and handling of carbon monoxide will be omitted. Furthermore, the presence of carbon dioxide in the reaction medium will provide the conditions for CXL, which will increase the reaction rate of the hydroformylation reaction.

It is the intention of the present invention to provide a syngas generating apparatus based on solid oxide electrolysis cells (SOECs) in combination with a reverse water gas shift (RWGS) reactor, which can generate syngas for hydroformylation plants. The raw materials for generating the syngas will be H₂O and CO₂.

Regarding prior art, U.S. Pat. No. 8,568,581 discloses a hydroformylation process using a traditional electrochemical cell, not a solid oxide electrolysis cell (SOEC) or an SOEC stack, for preparation of the synthesis gas to be used in the process. Water is introduced in a first (anode) compartment of the cell, and CO₂ is introduced into the second (cathode) compartment of the cell followed by alkene and catalyst addition to the cell, and the cathode induces liquid phase hydroformylation when an electrical potential is applied between the anode and the cathode.

In WO 2017/014635, a method for electrochemically reducing carbon dioxide is described. The method involves the conversion of CO₂ into one or more platform molecules such as syngas, alkenes, alcohols (including diols), aldehydes, ketones and carboxylic acids, and also conversion of CO₂ into i.a. CO, hydrogen and syngas. The method does not, however, include preparation of low-module syngas for hydroformylation.

US 2014/0291162 discloses a multi-step method for preparation of various compounds, such as aldehydes, by electrolysis of previously prepared CO₂ and/or CO and steam. The method includes i.a. heat transfer from a heating means towards a proton-conductive electrolyser comprising a proton-conducting membrane which is arranged between the anode and the cathode.

WO 2007/109549 discloses a hydroformylation process, which comprises reacting an olefin with CO and H₂ over a hydroformylation catalyst in a liquid that has been volumetrically expanded with a compressed gas, such as supercritical CO₂.

In WO 2008/124538, a CO₂ negative method of preparing renewable hydrogen and trapping CO₂ from the air or gas streams is described. Direct current renewable electricity is provided to a water electrolysis apparatus with sufficient voltage to generate hydrogen and hydroxide ions at the cathode and protons and oxygen at the anode. These products are separated and sequestered, and the base is used to trap CO₂ from the air or gas streams as bicarbonate or carbonate salts. These carbonate salts, hydrogen and trapped CO₂ in turn can be combined in a variety of chemical and electrochemical methods to create valuable carbon-based materials made from atmospheric CO₂. The net effect of all processes is the generation of renewable hydrogen from water and a reduction of CO₂ in the atmosphere or in gas destined to enter the atmosphere.

WO 2008/124538 is very specific about the source of CO₂ as well as about the source of electricity used for the electrolysis as opposed to the present invention. Furthermore, in WO 2008/124538, electrolysis is only used to produce hydrogen while, in the present invention, CO₂ is converted to CO by high-temperature electrolysis.

Finally, US 2011/0253550 discloses a method for producing a synthetic material, where water is converted into H₂ and O₂ using high-temperature electrolysis. Depending on how the catalytic process is carried out, the mixture of water vapor, CO₂ and H₂ can additionally be converted catalytically into functionalized hydrocarbons, such as aldehydes. This publication is very unspecific and does not define the concept of high-temperature electrolysis, neither in terms of temperature range nor in terms of the kind(s) of equipment being usable for the purpose.

### DETAILED DESCRIPTION

Now it has turned out that the above-described elements of risk in relation to syngas can effectively be counteracted by generating the syngas, which is necessary for hydroformylation plants, in an apparatus based on solid oxide electrolysis cells (SOECs) or SOEC stacks. A solid oxide electrolysis cell is a solid oxide fuel cell (SOFC) run in reverse mode, which uses a solid oxide electrolyte to produce e.g. oxygen and hydrogen gas by electrolysis of water. The SOEC technology is an advantageous alternative to low-temperature electrolysis technologies because of its high efficiency. The turn-on/turn-off of the apparatus is very swift, which is a further advantage.

In practice it will usually be desirable to operate the SOEC stack at less than full conversion, and therefore the product gas from the SOEC or SOEC stack will contain H₂ and H₂O.

In one embodiment of the invention, the raw product gas from the SOEC or SOEC stack is cooled, whereby most of the steam will condense, so that it can be separated from the gas stream as liquid water in a separator. The product gas may be further dried, e.g. over a drying column, if desired. The product gas, now containing H₂ as the main component, is then transferred to the RWGS reactor which is co-fed with CO₂. This embodiment has the advantage of pushing the equilibrium in the RWGS reaction in the direction of formation of CO and H₂O.

In another embodiment of the invention, the raw product gas from the SOEC or the SOEC stack is not cooled, but rather transferred directly to the RWGS reactor which is co-fed with CO₂. This embodiment has the advantage that the preferred operation temperatures of the SOEC or SOEC stack and the RWGS reactor are close lying; e.g. 700° C.

After the RWGS reactor, the syngas will contain H₂, CO, H₂O, and CO₂. By cooling the gas, most of the H₂O can be brought to condense and thus easily be separated from the gas. Further drying of the syngas may be carried out by using e.g. a drying column.

The separation of CO₂ from the reactive components CO and H₂ is more complicated and costly than the separation of water from the product gas. It can be done by using a PSA (pressure swing adsorption) unit, but such a unit is expensive. However, the presence of CO₂ in the hydroformylation reaction actually is an advantage: The hydroformylation reaction is carried out in a liquid medium. Pressurizing this liquid with CO₂ leads to the so-called CO₂-expanded liquid (CXL). It has been described in the literature (see Fang et al. Ind. Eng. Chem. Res. 46 (2007) 8687-8692 and references therein) that CXL media alleviates mass transfer limitations in the hydroformylation reaction and increases the solubility of the reactant gases in the CXL medium compared to the neat liquid medium. As a result of this, the rate of the hydroformylation reaction may be increased by up to a factor of four in CXL-media compared to neat organic solvents. Furthermore, the n/iso ratio (the ratio between linear and branched aldehydes) may be improved by using a CXL solvent compared to using the neat solvent as taught in U.S. Pat. No. 7,365,234.

Therefore, the present invention offers a way to provide a syngas with the appropriate H₂/CO ratio while at the same time providing the CO₂ needed for obtaining a CO₂-expanded liquid reaction medium for the hydroformylation process.

An example of an olefin used for the hydroformylation reaction is 1-octene, but in principle any olefin may be used according to the present invention. An example of a liquid solvent for the hydroformylation reaction is acetone, but a long range of other organic solvents may be used.

So it is the intention of the present invention to provide a syngas-generating apparatus based on a combination of solid oxide electrolysis cells and an RWGS reactor, which can generate syngas for hydroformylation plants. The raw materials for generating the syngas will be CO₂ and H₂O.

A solid oxide electrolysis cell system comprises an SOEC core wherein the SOEC stack is housed together with inlets and outlets for process gases. The feed gas or “fuel gas” is led to the cathode part of the stack, from where the product gas from the electrolysis is taken out. The anode part of the stack is also called the oxygen side because oxygen is produced on this side. In the stack, H₂ is produced from H₂O, which is led to the fuel side of the stack with an applied current, and excess oxygen is transported to the oxygen side of the stack, optionally using air, nitrogen, steam, or carbon dioxide to flush the oxygen side.

More specifically, the principle of producing H₂ by using a solid oxide electrolysis cell system consists in leading H₂O to the fuel side of an SOEC with an applied current to convert H₂O to H₂ and transport the oxygen surplus to the oxygen side of the SOEC. Air, nitrogen, steam, or carbon dioxide may be used to flush the oxygen side. Flushing the oxygen side of the SOEC has two advantages, more specifically (1) reducing the oxygen concentration and related corrosive effects and (2) providing means for feeding energy into the SOEC, operating it endothermic. The product stream from the SOEC contains a mixture of H₂ and H₂O, which—optionally after removal of water, e.g. by condensation—can be combined with CO₂ in the RWGS reaction.

If H₂O is fed into an SOEC stack, the output will be a mixture of H₂O and H₂. Steam will be electrochemically converted into gaseous hydrogen according to the following reaction:

$${H}_{2} O ( cathode ) - > {H}_{2} ( cathode ) + 1 / 2 {O}_{2} ( anode ) \qquad ( 1 )$$

The reverse water gas shift (RWGS) reaction takes place in the RWGS reactor which is fed with H₂ (and optionally H₂O) from the SOEC stack and CO₂:

$${H}_{2} + {CO}_{2} \text{<->} {H}_{2} O + CO \qquad ( 2 )$$

When pure H₂O is fed into the SOEC stack, the conversion X_{H2O} of H₂O to H₂ is given by Faraday's law of electrolysis:

$${X}_{{H}_{2} O} = \frac{{p}_{{H}_{2}}}{{p}_{{H}_{2}} + {p}_{{H}_{2} O}} = \frac{i \cdot {V}_{m} \cdot {n}_{cells}}{z \cdot {f}_{{H}_{2} O} \cdot F} \qquad ( 3 )$$

where p_{H2} is the partial pressure of H₂ at cathode outlet, P_{H2O} is the partial pressure of steam at cathode outlet, i is the electrolysis current, Vₘ is the molar volume of gas at standard temperature and pressure, n_{cells} is the number of cells in an SOEC stack, z is the number of electrons transferred in the electrochemical reaction, f_{H2O} is the flow of gaseous steam into the stack (at standard temperature and pressure), and F is Faraday's constant.

The equilibrium constant for the RWGS reaction, K_{RWGS}, is given by:

$${K}_{RWGS} = \frac{{p}_{CO} \cdot {p}_{{H}_{2} O}}{{p}_{{CO}_{2}} \cdot {p}_{{H}_{2}}} = exp ( - \frac{Δ G}{RT} ) \qquad ( 4 )$$

where ΔG is the Gibbs free energy of the reaction at the operating temperature, R is the universal gas constant, and T is absolute temperature.

The equilibrium constant, and therefore the extent to which electrochemically produced H₂ is used to convert CO₂ into CO, is temperature-dependent. For example, at 500° C., K_{RWGS}=0.195. At 600° C., K_{RWGS}=0.374. At 700° C., K_{RWGS}=0.619.

Thus, the present invention relates to a method for the generation of a gas mixture comprising carbon monoxide, carbon dioxide and hydrogen for use in hydroformylation plants, comprising the steps of:
evaporating water to steam,
feeding the steam to a solid oxide electrolysis cell (SOEC) or an SOEC stack at a sufficient temperature for the cell or cell stack to operate while supplying an electrical current to the cell or cell stack to effect a partial conversion of steam to hydrogen,
optionally removing some of or all the remaining steam from the raw product gas stream by cooling the raw product gas stream allowing for condensation of at least part of the steam as liquid water and separating the remaining product gas from the liquid,
utilizing the effluent SOEC gas comprising H₂ together with CO₂ from an external source as feed for a RWGS reactor in which the RWGS reaction takes place, converting some of the CO₂ and H₂ to CO and H₂O,
removing some of or all the remaining steam from the raw product gas stream by cooling the raw product gas stream allowing for condensation of at least part of the steam as liquid water and separating the remaining product gas from the liquid, and
using the gas mixture containing CO, CO₂ and H₂ for liquid phase hydroformylation utilizing carbon monoxide and hydrogen as reactants, while recycling CO₂ to the RWGS reactor.

Preferably the temperature, at which H₂ is produced by electrolysis of H₂O in the SOEC or SOEC stack, is around 700° C.

One of the great advantages of the method of the present invention is that the syngas can be generated with the use of virtually any desired CO/H₂ ratio, since this is simply a matter of adjusting the CO₂/H₂O ratio of the feed gases.

Another great advantage of the invention is, as already mentioned, that the syngas can be generated “on-site”, i.e. exactly where it is intended to be used, instead of having to transport the toxic and highly flammable syngas from the preparation site to the site of use.

Yet another advantage of the present invention is that if it is desired to switch between a low module syngas and pure H₂, this can be done using the same apparatus by simply bypassing the RWGS reactor when pure hydrogen is needed.

A further advantage of the present invention is that it provides a CO/H₂ stream diluted in CO₂ which enables the subsequent hydroformylation reaction to be carried out in a CO₂-expanded liquid (CXL) reaction medium. These advantages embrace higher reaction rates, improved selectivity (n/iso ratio) at mild conditions (lower temperature and lower pressure) compared to hydroformylation in a neat liquid media.

A still further advantage of the present invention is that syngas of high purity can be produced without in any way being more expensive than normal syngas, even though this desired high purity would prima facie be expected to entail increased production costs. This is because the purity of the syngas is largely determined by the purity of the CO₂/H₂O feed, and provided that a feed consisting of food grade or beverage grade CO₂ and ion-exchanged water is chosen, very pure syngas can be produced.

Embodiments of the invention is illustrated further in the examples which follow.

### Example 1—H₂O Electrolysis

An SOEC stack consisting of 75 cells is operated at an average temperature of 700° C. with pure steam fed to the cathode at a flow rate of 100 Nl/min steam (corresponding to a liquid water flow rate of approximately 80 g/min), while applying an electrolysis current of 50 A. Based on equation (3), the conversion of H₂O under such conditions is 26%, i.e. the gas exiting the cathode side of the stack is 26% H₂, 74% H₂O.

### Example 2—H₂O Electrolysis Combined with RWGS

An SOEC stack consisting of 75 cells is being operated at an average temperature of 700° C. with steam fed to the cathode with a total flow rate of 100 Nl/min, while applying an electrolysis current of 50 A. In the stack, steam is electrochemically converted into H₂ according to reaction (1) at a conversion of 52%. This effluent gas is fed directly from the SOEC to the RWGS reactor together with 100 Nl/min CO₂. The overall H₂O/CO₂ feed ratio is thus 50:50. The gas feeding the RWGS reactor will have the following composition: 0% CO, 50% CO₂, 26% H₂ and 24% H₂O. Due to the RWGS reaction, some of the hydrogen will be used to generate CO. The RWGS reactor is operated isothermally at 700° C. Therefore, the gas exiting the RWGS reactor will have the following composition: 10.7% CO, 39.3% CO₂, 15.3% H₂ and 34.7% H₂O. The ratio of CO:H₂ in the product gas is thus 1:1.43.

### Example 3—H₂O Electrolysis Combined with RWGS

This example is carried out as Example 2 except that the overall H₂O/CO₂ feed ratio is 41:59. The gas exiting the RWGS reactor will have the following composition: 13.2% CO, 45.8% CO₂, 13.0% H₂ and 28.0% H₂O. The ratio of CO:H₂ in the product gas is thus approximately 1:1.

### Example 4—H₂O Electrolysis Combined with RWGS

This example is carried out as Example 2 except that the effluent cathode gas from the SOEC stack is cooled, whereby steam condenses as liquid water which is taken out in a separator. The gas feeding the RWGS reactor will therefore have the following approximate composition: 0% CO, 50% CO₂, 50% H₂ and 0% H₂O. Due to the RWGS reaction, some of the hydrogen will be used to generate CO. The RWGS reactor is operated isothermally at 700° C. Therefore, the gas exiting the RWGS reactor will have the following approximate composition: 22% CO, 28% CO₂, 28% H₂ and 22% H₂O. The ratio of CO:H₂ in the product gas is thus 1:1.27.

1-3. (canceled)

4. A method for the generation of a gas mixture comprising carbon monoxide, carbon dioxide and hydrogen for use in hydroformylation plants, comprising the steps of:
evaporating water to steam,
feeding the steam to a solid oxide electrolysis cell (SOEC) or an SOEC stack at a sufficient temperature for the cell or cell stack to operate while supplying an electrical current to the SOEC or SOEC stack to effect a partial conversion of steam to hydrogen forming a raw product gas,
feeding CO₂ from an external source,
utilizing the raw product gas comprising H₂ together with the CO₂ as feed for a reverse water gas shift (RWGS) reactor in which the RWGS reaction takes place, converting some of the CO₂ and H₂ to CO and H₂O forming a RWGS product gas,
removing at least some of the remaining steam from the RWGS product gas stream by cooling the RWGS product gas stream allowing for condensation of at least part of the steam as liquid water and separating a remaining product gas from the liquid water, the remaining product gas comprising CO, CO₂ and H₂, and
using the remaining product gas for a liquid phase hydroformylation process utilizing the carbon monoxide and hydrogen as reactants, wherein the CO₂ in the remaining product gas is sufficient to form a CO₂-expanded liquid (CXL) reaction medium for the hydroformylation process.

5. The method according to claim 4, wherein the temperature, at which H₂ is produced by electrolysis of H₂O in the SOEC or SOEC stack, is around 700° C.

6. The method according to claim 4, comprising removing essentially all the remaining steam from the RWGS product gas stream by cooling the RWGS product gas stream allowing for condensation of the steam as liquid water and separating the remaining product gas from the liquid water.

7. The method according to claim 4, comprising removing at least some of remaining steam from the raw product gas stream by cooling the raw product gas stream allowing for condensation of at least part of the steam as liquid water and separating the dried raw product gas from the liquid, and utilizing the dried raw product gas in the reverse water gas shift (RWGS) reactor.

8. The method according to claim 4, comprising removing essentially all of remaining steam from the raw product gas stream by cooling the raw product gas stream allowing for condensation of essentially all of the steam as liquid water and separating the dried raw product gas from the liquid, and utilizing the dried raw product gas in the reverse water gas shift (RWGS) reactor.

9. The method according to claim 4, comprising removing essentially all the remaining steam from the RWGS product gas stream by cooling the RWGS product gas stream allowing for condensation of essentially all of the steam as liquid water and separating a remaining product gas from the liquid water, the remaining product gas comprising CO, CO₂ and H₂.

10. The method according to claim 4, wherein the RWGS reactor is a heated reactor.

11. The method according to claim 4, wherein the RWGS reactor is an adiabatic reactor.

12. The method according to claim 4, comprising flushing an oxygen side of the SOEC or SOEC stack.

13. The method according to claim 12, wherein flushing is with air, nitrogen, steam or carbon dioxide.

14. The method according to claim 4, comprising operating the SOEC or SOEC stack endothermic.

15. The method according to claim 4, comprising recycling CO₂ from the hydroformylation process to the RWGS reactor.

16. The method according to claim 4, comprising feeding excess CO₂ from the external source.

17. The method according to claim 4, comprising recycling CO₂ from the hydroformylation process to the RWGS reactor.

18. A method for the generation of a gas mixture comprising carbon monoxide, carbon dioxide and hydrogen for use in hydroformylation plants, comprising the steps of:
evaporating water to steam,
feeding the steam to a solid oxide electrolysis cell (SOEC) or an SOEC stack at a sufficient temperature for the cell or cell stack to operate while supplying an electrical current to the SOEC or SOEC stack to effect a partial conversion of steam to hydrogen forming a raw product gas,
feeding excess CO₂ from an external source,
utilizing the raw product gas comprising H₂ together with the CO₂ as feed for a reverse water gas shift (RWGS) reactor in which the RWGS reaction takes place, converting some of the CO₂ and H₂ to CO and H₂O forming a RWGS product gas,
removing at least some of the remaining steam from the RWGS product gas stream by cooling the RWGS product gas stream allowing for condensation of at least part of the steam as liquid water and separating a remaining product gas from the liquid water, the remaining product gas comprising CO, CO₂ and H₂, and
using the remaining product gas for a liquid phase hydroformylation process utilizing the carbon monoxide and hydrogen as reactants, while utilizing the CO₂ to form a CO₂-expanded liquid (CXL) reaction medium for the hydroformylation process.

19. A method for the generation of a gas mixture comprising carbon monoxide, carbon dioxide and hydrogen for use in hydroformylation plants, comprising the steps of:
evaporating water to steam,
feeding the steam to a solid oxide electrolysis cell (SOEC) or an SOEC stack at a sufficient temperature for the cell or cell stack to operate while supplying an electrical current to the SOEC or SOEC stack to effect a partial conversion of steam to hydrogen forming a raw product gas,
feeding CO₂ from an external source,
utilizing the raw product gas comprising H₂ together with the CO₂ as feed for a reverse water gas shift (RWGS) reactor in which the RWGS reaction takes place, converting some of the CO₂ and H₂ to CO and H₂O forming a RWGS product gas,
removing at least some of the remaining steam from the RWGS product gas stream by cooling the RWGS product gas stream allowing for condensation of at least part of the steam as liquid water and separating a remaining product gas from the liquid water, the remaining product gas comprising CO, CO₂ and H₂, and
using the remaining product gas for a liquid phase hydroformylation process utilizing the carbon monoxide and hydrogen as reactants, while utilizing the CO₂ to form a CO₂-expanded liquid (CXL) reaction medium for the hydroformylation process
recycling CO₂ from the hydroformylation process to the RWGS reactor.
